(19) United States
(12) Patent Application Publication
(10) Pub. No.: US 2025/0207127 A1
(43) Pub. Date: Jun. 26, 2025

(54) PHYTOECDYSONES AND/OR 20-HYDROXYECDYSONE DERIVATIVES IN COMBINATION WITH AN ACTIVE INGREDIENT FOR RESTORING SMN EXPRESSION, FOR USE IN THE TREATMENT OF SPINAL MUSCULAR ATROPHY

(71) Applicants: BIOPHYTIS, PARIS (FR); SORBONNE UNIVERSITÉ, PARIS (FR); UNIVERSITÉ PARIS CITÉ, PARIS (FR); INSTITUT NATIONAL DE LA SANTÉ ET DE LA RECHERCHE MÉDICALE, PARIS Cedex 13 (FR)

(72) Inventors: Mathilde LATIL, PARIS (FR); Cynthia BEZIER, PARIS (FR); Pierre DILDA, PARIS (FR); René LAFONT, PARIS (FR); Stanislas VEILLET, SAVIGNY SUR ORGE (FR); Frederic CHARBONNIER, PARIS (FR); Olivier BIONDI, BOISSISE LA BERTRAND (FR)

(21) Appl. No.: 18/708,830

(22) PCT Filed: Nov. 4, 2022
(86) PCT No.: PCT/EP2022/080839

§ 371 Date: Dec. 30, 2024

(30) Foreign Application Priority Data
Nov. 10, 2021 (FR) FR2111920

(51) Int. Cl. C12N 15/113; A61K 31/575; A61K 36/28; A61K 38/17; A61P 21/00

(52) U.S. Cl. CPC C12N 15/113; A61K 31/575; A61K 36/28; A61K 38/1709; A61P 21/00; C12N 2310/11; C12N 2310/315; C12N 2310/3341; C12N 2320/31

(57) ABSTRACT

The invention relates to at least one phytoecdysone and/or at least one semi-synthetic derivative of 20-hydroxyecdysone, and at least one active ingredient having the ability to increase the production of functional SMN protein, for use in a combination therapy In the treatment of spinal muscular atrophy in mammals.

### TECHNICAL FIELD OF THE INVENTION

The invention relates to the use of phytoecdysones and/or semi-synthetic derivatives of 20-hydroxyecdysone, in combination with an active ingredient aimed at restoring the expression of the SMN protein, for the treatment of spinal muscular atrophy.

### PRIOR ART

Neuromuscular diseases are characterised by an alteration in the functioning of motor units, composed of motor neurons, neuromuscular junctions and skeletal muscles. Regardless of the origin of the disease, nervous as in spinal muscular atrophy or amyotrophic lateral sclerosis, or muscular, all cause an alteration of the motor function of patients, which can range from disability to premature death when vital muscles are affected.

Among these neuromuscular diseases, two are described as specifically affecting motor neurons: infantile spinal muscular atrophy (or SMA), whose symptoms appear in childhood, and amyotrophic lateral sclerosis (or ALS), whose symptoms appear in adulthood. These two neurodegenerative diseases, with different causes and clinical manifestations, have in common a progressive muscular denervation, responsible for a muscular atrophy (Al-Chalabi and Hardiman, 2013; Crawford and Pardo, 1996).

Spinal muscular atrophies represent the most common cause of infant mortality of genetic origin with a prevalence of 1/6,000 to 1/10,000 births (Crawford and Pardo, 1996). Three main types of severity are described, depending on the age of onset of symptoms and the progression of clinical damage, ranging from type 1, the most severe, to type 3, whose life expectancy can be greater than 40 years. Patients with SMA have a symmetrical damage to the skeletal muscles by atrophy of muscle fibres which are isolated or grouped into fascicles. Almost all SMA are predominantly proximal, that is to say affecting the muscles of the trunk and close to the trunk. Gradually, the motor deficit extends, firstly, to the muscles of the lower limbs, then secondly, to the muscles of the upper limbs, preferentially affecting the extensor muscles. The gene responsible for SMA was identified in 1995 on chromosome 5 and was named SMN for “survival of motor neurons” (Lefebvre et al., 1995).

Although having a great clinical heterogeneity, genetic analyzes have been able to demonstrate that all forms of SMA are caused by the homozygous alteration of the SMN1 (Survival Of Motor Neurons) telomeric gene, preventing the production of the Smn protein, inducing the degeneration of the motor neurons, muscle atrophy and weakness. In the human genome, there is an inverted centromeric copy of this gene, the SMN2 gene, which can be found in several copies (Lorson et al., 1998), but which only allows partially compensating for the loss of function of the SMN1 gene. Indeed, SMN2 has 5 nucleotide differences with SMN1, including one at exon 7, favouring its excision by splicing in 90% of the mRNAs produced by the SMN2 gene. This alternative splicing leads to the production of a truncated and unstable SMNA7 protein. Thus, only 10% of the proteins produced by the SMN2 gene are complete and functional (Lefebvre et al., 1997; Vitte et al., 2007). A link has been demonstrated between the number of copies of the SMN2 gene, their expression level and the severity of the disease.

Several therapeutic strategies, at different stages of development, are explored in SMN1-related proximal spinal muscular atrophy. Certain studied strategies aim at increasing the quantity of functional SMN protein, either by modifying the maturation of the SMN2 messenger RNA so that it reintegrates the missing exon 7 (Nusinersen, Risdiplam, Branaplam), or by providing the SMN1 gene by gene therapy (Zolgensma®).

Others aim at slowing the progression of the disease by protecting motor neurons or by improving the functioning of neuromuscular junctions (Salbutamol, Pyridostigmine), or muscular performance (SRK-015, physical training). Nevertheless, the majority of therapeutic approaches tested in SMA aim at increasing the expression levels of the SMN protein, either locally at the central nervous system and/or more generally at other organs, in a peripheral manner.

Gene therapy to deliver the SMN1 gene using adeno-associated viral (AAV) vectors has shown significant beneficial effects in preclinical studies in mice, and in clinical trials in humans (Mendell et al. 2017; Passini et al., 2010; Lowes et al., 2018). Other approaches focusing on the modulation of SMN2 splicing have also proven their effectiveness (Naryshkin et al., 2014; Palacino et al., 2015; Finkel et al., 2016°; Finkel et al., 2017). These very positive results enabled the regulatory authorisation of certain molecules.

Three treatments are currently available in the SMA:
The first treatment to be authorised for SMA was Spinraza® (nusinersen). This treatment received the marketing authorisation (MA) in December 2016 in the United States and in June 2017 in Europe. This is an antisense oligonucleotide (ASO) developed with the aim of increasing the production of functional SMN protein by acting on the maturation (splicing) of the SMN2 gene. Antisense oligonucleotides do not cross the blood-brain barrier, nusinersen must be administered regularly intrathecally, allowing a re-expression of the SMN protein in motor neurons, with a real clinical benefit, but of variable importance depending on the type of SMA and age of start of treatment. Sequences of antisense oligonucleotides having the ability to modify the splicing of the SMN2 gene are given in applications WO2007/002390 and WO2018/014041.
More recently, Zolgensma@ (Onasemnogène abeparvovec or AVXS-101), a gene therapy product aimed at delivering the SMN1 gene using a viral vector (AAV) was authorised (MA in May 2019 in the United States and a conditional MA in May 2020 in Europe). The single administration thereof is easier, because it is intravenously.
Finally, Evrysdi® (Risdiplam or RO7034067) was authorised even more recently (MA in August 2020 in the United States and in March 2021 in Europe). It is a small molecule which acts on the maturation of the SMN2 gene. The administration thereof is daily orally or by feeding tube.

Although the restoration of the SMN gene has allowed an unprecedented improvement in functional measures in patients as well as their motor function, significant deficits persist in patients with SMA after treatment, and this, even if the intervention is precocious (Mercuri et al., 2018; Finkel et al., 2017; Baranello et al., 2018).

Preclinical studies showed that these post-treatment deficits were found in SMA mouse models, where the treated animals had reduced life expectancy, a deficit in body weight and muscle mass and function compared to healthy animals (Passini et al., 2010; Hua et al, 2010, 2011; Feng et al., 2016).

In addition, there are still other limitations and significant concerns regarding these treatments. For example, nusinersen must be administered by intrathecal injection, which is highly invasive, several times a year. This method of administration is very difficult and sometimes impossible for patients having undergone surgeries for scoliosis, which excludes nusinersen as a therapeutic option for these patients. In addition, intrathecal administration allows a specific distribution to the central nervous system, which means that not all symptoms can be fully covered. On this last aspect, onasemnogene benefits from an advantage, because it is administered intravenously, and allows a systemic distribution. Nevertheless, the question of the bioavailability of the AAV9 serotype is still open, as well as the fact that the long-term transgene expression should be limited to the post-mitotic cells such as neurons (Chaytow et al., 2021). Although onasemnogene is advertised as a therapy requiring only a single injection, it remains unclear whether the treatment will last over the patients' lifetimes, or whether boosters will be necessary.

Risdiplam, for its part, is a systemic therapy, which is less invasive, by daily oral administration. However, to the extent that risdiplam targets the splicing machinery, it may also affect other transcripts, leading to unknown and uncontrollable non-targeted side effects. Indeed, it has been described that risdiplam appears to have an effect on a regulator of the cell division at high concentrations, raising fears of oncogenic side effects (Ratni et al., 2018).

Phytoecdysones represent an important family of polyhydroxylated sterols. These molecules are produced by various species of plants (ferns, gymnosperms, angiosperms) and participate in their defence against insect pests. The majority phytoecdysone in the plant kingdom is 20-hydroxyecdysone.

Patent FR 3 021 318 discloses that phytoecdysones, and more particularly 20-hydroxyecdysone (20E), have been the subject of numerous pharmacological studies. These studies have highlighted the antidiabetic and anabolic properties of this molecule. Its stimulating effects on protein synthesis in muscles are observed in rats in vivo (Syrov et al., 2000; Tóth et al., 2008; Lawrence et al., 2012) and on C2C12 mouse myotubes in vitro (Gorelick-Feldman et al., 2008). Some of the effects described above in animal models have been found in clinical studies, which are still few in number. Thus, 20E promotes the increase in muscle mass in young athletes (Simakin et al., 1988).

Finally, French patent FR 19 02726 further describes the use of phytoecdysones and semi-synthetic derivatives thereof for the treatment of neuromuscular diseases, in particular infantile spinal muscular atrophy and amyotrophic lateral sclerosis (Latil et al., 2020).

The above elements indicates that, although the therapies that increase SMN protein expression may have significant effects on disease progression and patient quality of life, it remains useful to find complementary therapeutic approaches which would allow reducing the administration doses of the already authorised treatments, or their frequency of administration or finally improving the functional gains in order to further reduce the burden of the disease.

### Presentation of the Invention

The present invention aims at providing a treatment for spinal muscular atrophy, said treatment being improved relative to the existing treatments.

The inventors unexpectedly discovered that phytoecdysones (in particular 20E and semi-synthetic derivatives thereof) had a beneficial and synergistic effect when used in combination therapy with an active ingredient having the ability to increase the production of functional SMN protein, in mammals affected by spinal muscular atrophy. Indeed, the use of phytoecdysones and/or at least one semi-synthetic derivative of 20-hydroxyecdysone, in combination with a treatment using an active ingredient having the ability to increase the production of functional SMN protein, improves, by synergy of these elements, the motor and functional performance as well as the survival and the weight of animals affected by spinal muscular atrophy (SMA).

To this end, the present invention aims for at least one phytoecdysone and/or at least one semi-synthetic derivative of 20-hydroxyecdysone, and at least one active ingredient having the ability to increase the production of functional SMN protein, for use in combination therapy in the treatment of spinal muscular atrophy.

In particular embodiments of the present invention, the invention relates to at least one phytoecdysone and/or at least one semi-synthetic derivative of 20-hydroxyecdysone, and at least one active ingredient having the ability to increase the production of functional SMN protein, for their use in combination therapy in the treatment of a motor neuron disease, said disease participating in SMA.

In the context of the present application, the term “combination therapy” designates the co-administration of at least two biologically active agents. In the case of the present invention, a first active agent is selected from phytoecdysones or semi-synthetic derivatives of 20-hydroxyecdysone or is a composition comprising at least one phytoecdysone and/or at least one semi-synthetic derivative of 20-hydroxyecdysone, a second active agent being the active ingredient having the ability to increase the production of functional SMN protein. The combination therapy may comprise a single formulation or several formulations. The co-administration can be performed simultaneously or sequentially. The co-administration may be performed by the same route of administration or by different routes of administration. It is considered as a combination therapy as long as the effects of the two (or more) agents overlap in the subject to obtain additional, additive, or synergistic clinical effects.

In particular embodiments, the invention further meets the following characteristics, implemented separately or in each of the technically operative combinations thereof.

In particular embodiments, said at least one phytoecdysone and/or said at least one semi-synthetic derivative of 20-hydroxyecdysone, and said at least one active ingredient having the ability to increase the production of functional SMN protein are co-administered.

In particular embodiments, said at least one phytoecdysone and/or said at least one semi-synthetic derivative of 20-hydroxyecdysone, and said at least one active ingredient having the ability to increase the production of functional SMN protein are co-administered within the same composition.

In particular embodiments, said at least one phytoecdysone and/or said at least one semi-synthetic derivative of 20-hydroxyecdysone are administered orally or systemically to the mammal, and said at least one active ingredient having the ability to increase the production of functional SMN protein is administered to the mammal orally and/or intrathecally and/or intravenously.

For oral administration, said at least one phytoecdysone and/or said at least one semi-synthetic derivative of 20-hydroxyecdysone and/or the active ingredient having the ability to increase the production of functional SMN protein, is preferably incorporated to an pharmaceutically acceptable formulation which can be administered orally.

Increasing the production of SMN protein can be obtained by gene therapy or by therapy of the gene. According to one implementation of gene therapy, the active ingredient having the ability to increase the production of SMN protein aims at providing the SMN1 gene. According to one implementation of a gene therapy, the active ingredient having the ability to increase the production of SMN protein is either a small molecule which acts on the maturation of the SMN2 gene, or an antisense oligonucleotide (ASO) developed in the aim of increasing the production of functional SMN protein by acting on the splicing (maturation) of the SMN2 gene. In the latter case, the ASO modulates the splicing of SMN2 which allows increasing the inclusion of exon 7 in the SMN2 mRNA and thus allows increasing the number of generated SMN proteins having the amino acids corresponding to exon 7 and therefore corresponding to a functional SMN protein.

Thus, in particular embodiments of the present invention, the active ingredient has the ability to increase the production of functional SMN protein by gene therapy or by therapy of the gene.

In particular embodiments of the present invention, the active ingredient has the ability to increase the production of functional SMN protein by providing the SMN1 gene.

In particular embodiments of the present invention, the active ingredient has the ability to increase the production of functional SMN protein by action on the maturation of the SMN2 gene.

In particular embodiments, the active ingredient having the ability to increase the production of functional SMN protein is an antisense oligonucleotide (ASO). This ASO preferably has a sequence of 10 to 30 nucleotides, more preferably 12 to 30 nucleotides, preferably 12 to 25 nucleotides or even preferably 15 to 20 nucleotides. According to preferred embodiments of the present invention, the ASO has a sequence of 18 nucleotides.

In particular embodiments of the present invention, the ASO has the capacity to induce the inclusion of exon 7 in the sequence of the SMN2 gene.

In particular embodiments of the present invention, the ASO is complementary to at least 50%, preferably at least 60%, preferably at least 70%, more preferably at least 80%, most preferably at least 90%, preferably at least 95%, most preferably 98%, preferably 100%, of the sequence of the nucleic acid encoding the pre-mRNA of the human SMN2 gene.

In particular embodiments of the present invention, the ASO is complementary to at least 50%, preferably at least 60%, preferably at least 70%, more preferably at least 80%, most preferably at least 90%, preferably at least 95%, most preferably 98%, preferably 100%, of a sequence belonging to intron 6, to intron 7 or to a portion of exon 7 and a portion of an intron adjacent to exon 7, of the nucleic acid encoding the pre-mRNA of the human SMN2 gene. In this manner, exon 7 is included in the SMN2 mRNA, which allows obtaining a functional SMN protein.

The sequence of the nucleic acid encoding the pre-mRNA of the human SMN2 gene is available on Genbank under the reference NG_008728, version NG_008728.1.

In particular embodiments, the ASO is a sequence identical or similar to at least 50% with the sequence SEQ ID NO: 1 given in the sequence listing filed with the present application (5′-TCACTTTCATAATGCTGG-3′), preferably identical or similar to at least 60%, preferably at least 70%, more preferably at least 80%, most preferably at least 90% and preferably identical or similar to 100%. The sequence SEQ ID NO: 1 is complementary to 18 bases of intron 7 (bases 10 to 27) of the SMN2 gene.

In particular embodiments, the ASO has a sequence selected from the sequences SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 25, SEQ ID NO: 26, SEQ ID NO: 27, SEQ ID NO: 28, SEQ ID NO: 29. These sequences allow ASO to target intron 7 of the SMN2 gene.

When the ASO sequence has a percentage of identity or similarity which is less than 100% relative to one of the sequences listed above, it may have insertions, deletions and/or substitutions relative to this reference sequence.

The percentage of identity between two ASO sequences is determined by comparing the two optimally aligned sequences, through a comparison window. The portion of the sequence of an ASO in the comparison window can thus comprise additions or deletions relative to the reference sequence so as to obtain optimal alignment between the two sequences.

The percentage of identity is calculated by determining the number of positions for which a nucleic base is identical in the two compared sequences, then by dividing this number of positions by the total number of positions in the comparison window, the obtained number being multiplied per hundred.

The ASO is preferably composed of a phosphodiester backbone. It may have various modifications to its backbone and/or its chemical structure in order to increase its stability and/or its affinity for RNA and/or to have a significant advantage in terms of pharmacokinetics.

In particular embodiments, the ASO includes at least one modification selected from:
a modification at the phosphate group such as a phosphorothioate, or a methylphosphanate, or a phosphoroamidate,
a chemical modification in the 2′ position of the ribose, such as a 2′O-methyl (2′OMe) or a 2′O-methoxyethyl (2′MOE) or a 2′ Fluoro (2′ F),
at least one modification of nucleobases such as the methylation of 5′ methylcytosine, 5-methyluridine/ribothymidine, or “G-clamp” type pyrimidine,
at least one substantial change in the structure of the sugar, leading to a variety of molecules, such as morpholinos (PMO for “phosphoroamidate morpholino oligomer”) or the peptide nucleic acids (PNA) or constrained type oligonucleotides (LNA for “locked nucleic acid” or cEt for “2′-4′-constrained ethyl” or tc-DNA for tricyclo-DNA).

In particular embodiments, the ASO is of a sequence which is at least 50% identical or similar with the sequence SEQ ID NO: 23, preferably identical or similar to at least 60%, preferably at least 70%, more preferably at least 80%, most preferably at least 90% and preferably identical or similar to 100%. The sequence SEQ ID NO: 23 corresponds to the sequence SEQ ID NO: 1 with 2′-O-methoxyethyl on the 2′ carbon atom of the deoxyribose for each base, with a phosphorothioate backbone, and with 5-methyl cytosines instead of cytosines. The phosphothioate backbone advantageously improves the stability of the ASO, the 5-methyl cytosines advantageously allow making the ASO less sensitive to nucleases and the 2′-O-methoxyethyl on the 2′ carbon atom of the deoxyribose advantageously allows reducing the immune response induced by the administration of ASO.

In particular embodiments of the present invention, ASO is administered at a dose comprised between 0.01 and 10 mg per kilogram in humans. Preferably this dose is administered per day or per week.

For the use thereof in the present invention, the phytoecdysones and the semi-synthetic derivatives of 20-hydroxyecdysone are advantageously purified to the pharmaceutical grade.

A phytoecdysone which can be used according to the invention is for example 20-hydroxyecdysone.

To this end, according to particular embodiments of the present invention, said at least one phytoecdysone is 20-hydroxyecdysone.

20-hydroxyecdysone and the semi-synthetic derivatives thereof are advantageously purified to the pharmaceutical grade.

The used 20-hydroxyecdysone is preferably in the form of a plant extract rich in 20-hydroxyecdysone or of a composition comprising 20-hydroxyecdysone as active agent. Plant extracts rich in 20-hydroxyecdysone are, for example, extracts of Stemmacantha carthamoides (also called Leuzea carthamoides), Cyanotis arachnoidea and Cyanotis vaga.

The obtained extracts are preferably purified to the pharmaceutical grade.

In one embodiment, 20-hydroxyecdysone is in the form of a plant extract or a portion of a plant, said extract including at least 95%, and preferably at least 97%, of 20-hydroxyecdysone. Said plant is preferably selected from the plants containing at least 0.5% of 20-hydroxyecdysone by dry weight of said plant. Said extract is preferably purified to the pharmaceutical grade.

Said extract is subsequently called BIO101. It remarkably includes between 0 and 0.5%, by dry weight of the extract, of impurities, such as minor compounds, likely to affect the safety, availability or effectiveness of a pharmaceutical application of said extract.

The plant from which BIO101 is produced is preferably selected from Stemmacantha carthamoides, Cyanotis arachnoidea and Cyanotis vaga.

The semi-synthetic derivatives of 20-hydroxyecdysone are obtained by semisynthesis and can in particular be obtained in the manner described in European patent application No. EP 15732785.9.

In a particular embodiment, the phytoecdysones are administered at a dose comprised between 3 and 15 milligrams per kilogram per day in humans. The term “phytoecdysones” means here both phytoecdysones in general and 20-hydroxyecdysone (in particular in extract form) and the semi-synthetic derivatives thereof.

Preferably, phytoecdysones are administered at a dose of 200 to 1000 mg/day, in one or more doses, in an adult human, and a dose of 5 to 350 mg/day, in one or more doses, in human child or infant. The term “phytoecdysone” means here both phytoecdysones in general and 20-hydroxyecdysone (in particular in extract form) and the semi-synthetic derivatives thereof.

In particular embodiments of the present invention, said at least one semi-synthetic derivative of 20-hydroxyecdysone is selected from:
a compound of general formula (I):

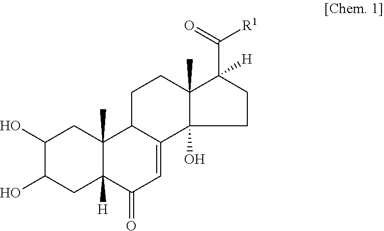

wherein:
R¹ is selected from: a (C₁-C₆) W(C₁-C₆) group; a (C₁-C₆) W(C₁-C₆) W(C1-C₆) group; a (C₁-C₆) W(C₁-C₆) CO₂ (C₁-C₆) group; a (C₁-C₆) A group, A representing a heterocycle optionally substituted by a group of the OH, OMe, (C₁-C₆), N(C₁-C₆), CO₂ (C₁-C₆) type; a CH₂Br group; W being a heteroatom selected from N, O and S, preferably O and even more preferably S°; and,
a compound having formula (II):

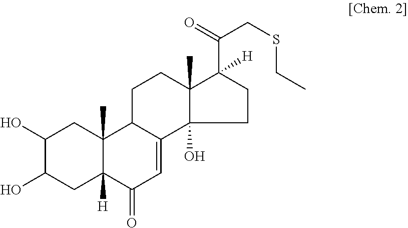 [SMILES: CCSCC(=O)[C@H]1CC[C@@]2(O)C3=CC(=O)[C@@H]4CC(O)C(O)C[C@]4(C)C3CC[C@]12C]

Within the scope of the present invention, the term “(C₁-C₆)” means any alkyl group of 1 to 6 linear or branched carbon atoms in particular, methyl, ethyl, n-propyl, iso-propyl, n-butyl, iso-butyl, sec-butyl, t-butyl, n-pentyl, n-hexyl. Advantageously it is a methyl, ethyl, iso-propyl or t-butyl group, in particular a methyl or ethyl group, more particularly a methyl group.

Within the scope of the present invention, the term “heterocycle” preferably means a cycle comprising 5 or 6 atoms including one or two heteroatoms (O, S or N), the remaining atoms being carbon atoms.

In a preferred embodiment of the present invention, in general formula (I):
R¹ is selected from: a (C₁-C₆) W(C₁-C₆) group; a (C₁-C₆) W(C₁-C₆) W(C₁-C₆) group; a (C₁-C₆) W(C₁-C₆) CO₂ (C₁-C₆) group; a (C₁-C₆) A group, A representing a heterocycle optionally substituted by a group of the OH, OMe, (C₁-C₆), N(C₁-C₆), CO₂ (C₁-C₆) type;
W being a heteroatom selected from N, O and S, preferably O and more preferably S.

In particular embodiments of the present invention said at least one semi-synthetic derivative of 20-hydroxyecdysone is a compound selected from the following compounds:
no. 1: (2S,3R,5R,10R,13R,14S,17S)-2,3,14-trihydroxy-10,13-dimethyl-17-(2-morpholinoacetyl)-2,3,4,5,9,11,12,15,16,17-decahydro-1H-cyclopenta[a]phenanthren-6-one,
no. 2: (2S,3R,5R,10R,13R,14S,17S)-2,3,14-trihydroxy-17-[2-(3-hydroxypyrrolidin-1-yl) acetyl]-10,13-dimethyl-2,3,4,5,9,11,12,15,16,17-decahydro-1 H-cyclopenta[a]phenanthren-6-one;
no. 3: (2S,3R,5R,10R,13R,14S,17S)-2,3,14-trihydroxy-17-[2-(4-hydroxy-1-piperidyl) acetyl]-10,13-dimethyl-2,3,4,5,9,11,12,15,16,17-decahydro-1H-cyclopenta[a]phenanthren-6-one;
no. 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
no. 5: (2S,3R,5R,10R,13R,14S,17S)-17-[2-(3-dimethylaminopropyl (methyl)amino) acetyl]-2,3,14-trihydroxy-10,13-dimethyl-2,3,4,5,9,11,12,15,16,17-decahydro-1H-cyclopenta[a]phenanthren-6-one;
no. 6:2-[2-oxo-2-[(2S,3R,5R,10R, 13R,14S,17S)-2,3,14-trihydroxy-10,13-dimethyl-6-oxo-2,3,4,5,9,11,12,15,16,17-décahydro-1H-cyclopenta[a]phenanthren-17-yl]ethyl]ethylsulfanylacetate;
no. 7: (2S,3R,5R,10R,13R,14S,17S)-17-(2-ethylsulfanylacetyl)-2,3,14-trihydroxy-10,13-dimethyl-2,3,4,5,9,11,12,15,16,17-decahydro-1H-cyclopenta[a]phenanthren-6-one;
no. 8: (2S,3R,5R,10R,13R,14S, 17S)-2,3,14-trihydroxy-17-[2-(2-hydroxyethylsulfanyl) acetyl]-10,13-dimethyl-2,3,4,5,9,11,12,15,16,17-decahydro-1H cyclopenta[a]phenanthren-6-one.

According to another aspect, the present invention aims at a composition comprising:
at least one phytoecdysone and/or at least one semi-synthetic derivative of 20-hydroxyecdysone,
at least one active ingredient having the ability to increase the production of functional SMN protein,
for the use thereof in the treatment of a neuromuscular disease in mammals, in particular spinal muscular atrophy.

This use of the composition may meet one or more of the characteristics described above with reference to the use, in combination therapy, of said at least one phytoecdysone and/or at least one semi-synthetic derivative of 20-hydroxyecdysone, and of said at least one active ingredient having the ability to increase the production of functional SMN protein.

In embodiments, the composition is incorporated into a pharmaceutically acceptable formulation capable of being administered orally or intrathecally or systemically.

Within the scope of the present invention, the term “pharmaceutically acceptable” means that which is useful in the preparation of a pharmaceutical composition, which is generally safe, non-toxic and which is acceptable for a veterinary as well as human pharmaceutical use.

### BRIEF DESCRIPTION OF THE FIGURES

The invention will be better understood on reading the following description, given by way of non-limiting example, and made with reference to the figures which represent:

FIG. 1 represents the curve of the weight evolution of SMA mice (Smn^{Δ7/Δ7}; huSMN2^{+/-}) treated with BIO101 alone or with a mismatch ASO or with an ASO 10-27 alone, or with an ASO 10-27+BIO101 combination, from birth (PO) until the death of the mice. Here and in the remainder of the description, P corresponds to the number of days after birth (post-natal);

FIG. 2 is a Kaplan-Meier representation of the survival curves of SMA mice (Smn^{Δ7/Δ7}; huSMN2^{+/-}) treated with BIO101 alone or with a mismatch ASO or with an ASO 10-27 alone, or with an ASO 10-27+BIO101 combination, from birth (PO);

FIG. 3 represents the motor performances (movement capacities) evaluated by the open field test of SMA mice (Smn^{Δ7/Δ7}; huSMN2^{+/-}) treated with BIO101 alone or with a mismatch ASO or with an ASO 10-27 alone, or with an ASO 10-27+BIO101 combination, from birth (PO);

FIG. 4 represents the motor performances (muscle fatigability) evaluated by the grip-test of SMA mice (Smn^{Δ7/Δ7}; huSMN2^{+/-}) treated with BIO101 alone or with a mismatch ASO or with an ASO 10-27 alone, or with an ASO 10-27+BIO101 combination, from birth (PO).

For each of the figures, the results are presented as mean±SEM with *p<0.05, ** p<0.01, *** p<0.001 and **** p<0.0001.

In the remainder of the description, n corresponds to the sample size and p corresponds to the “p-value” used to quantify the statistical significance of a result. In addition, p=ns indicates the non-significance of the p-value.

### DESCRIPTION OF EMBODIMENTS

The invention will be described below in the particular context of one of the preferred, non-limiting fields of application thereof.

### 1. Description and Injection of the Antisense Oligonucleotide (ASO)

The antisense oligonucleotide (ASO) of sequence SEQ ID NO: 23 (5′-TCACTTTCATAATGCTGG-3′), of reference ISIS 396443, also known under the name ISIS-SMNRx, and called in the remainder of the description ASO 10-27, complementary to 18 bases (bases 10 to 27) of intron 7 of the SMN2 gene (targeting a site in intron 7, called ISS-N1, which represses the inclusion of exon 7 of the pre-messenger RNA (pre-mRNA) of the SMN2 gene) having modified nucleotides 2′-O-methoxyethyl on the 2′ carbon atom of deoxyribose for each base, with a phosphorothioate backbone and 5-methyl cytosines and the control ASO (denoted mismatch) of sequence SEQ ID NO°: 24 (5′-TTAGTTTAATCACGCTCG-3′) (Singh et al. 2006; Williams et al. 2009), were synthesised and purified as previously described (Baker et al. 1997, Hua et al. 2008; Passini et al., 2011). The oligonucleotides are resuspended at a concentration of 8 μg/μL in 0.9% NaCl and stored at −20° C.

On the day of injection, the ASO stock solution is diluted to the concentration of 2 μg/μL in 0.9% NaCl. Methylene blue 0.04% is added to the solution as an injection control. Newborn SMA mice receive a single dose of ASO 10-27 or ASO mismatch of 8 μg on the 1st post-natal day (PO) by unilateral intra-cerebro-ventricular injection using a Hamilton syringe and a 32G needle. The injection site is located 1 mm from the sagittal suture, between bregma and lambda, landmarks visible through the skin at PO. The needle is positioned perpendicular to the injection site on the surface of the skin and inserted approximately 3 mm deep to reach the lateral ventricle.

### 2. Co-Administration of BIO101

The complementary treatment BIO101 complexed with cyclodextrin is administered daily to the mice from the first post-natal day (PO) orally at a dose of 50 mg/kg using a pipette.

3. Biological Activity of BIO101 in Combination with ASO
a. Analysis of the Effects of Combined Treatment ASO 10-27+BIO101 on Weight and Survival

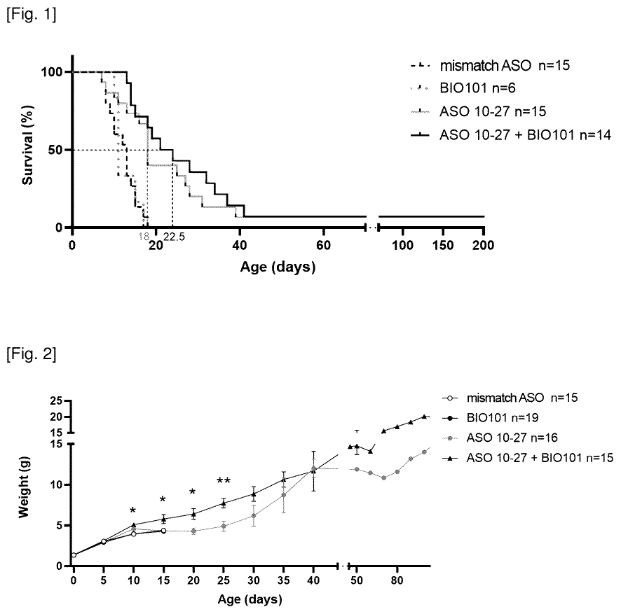

A severe SMA mouse model was used on a FVB/NRj genetic background, characterised by the invalidation of exon 7 of the murine Smn gene and expressing two copies of the human SMN2 transgene (Smn^{Δ7/Δ7}; huSMN2−/+) (Hsieh et al., 2000). The mice resulting from these crosses having the genotype “FVB/NRj-Smn^{Δ7/Δ7} huSMN2−/+2 copies” are described as “SMA” (Hsieh et al., 2000). SMA mice were treated with either BIO101 alone or a mismatch ASO or an ASO 10-27 alone, or with an ASO 10-27+BIO101 combined treatment starting from PO. Survival (FIG. 1) and weight

(FIG. 2) Mice were Analysed Daily.

The average lifespan of the mouse pups (FIG. 1) treated with BIO101 alone (11 days) is substantially identical to that of the mouse pups treated with mismatch ASO (13 days). As described in 2011 by Passini et al., the treatment with ASO 10-27 alone at a dose of 8 μg significantly increases the average lifespan of SMA mouse pups, with a median survival of 18 days (38% improvement in animal survival), compared to a median survival of 13 days in the mismatch ASO control group. When BIO101 treatment is administered in combination with ASO 10-27 the median survival increases to 22.5 days, i.e. an improvement of 73% in animal survival.

The weight of the mouse pups (FIG. 2) treated with BIO101 alone is comparable to that of the mouse pups treated with mismatch ASO throughout their life.

The treatment with ASO 10-27 alone does not increase the average weight of SMA mice relative to the control treatment with mismatch ASO at P7, but it significantly increases it by 9.9% at P8 and 13.9% at P9. The ASO 10-27+BIO101 combination induces a synergistic effect on the weight of SMA mouse pups with a significant increase of 11.3% at P7, 16.5% at P8 and 24.9% at P9.

At P10, the treatment with ASO 10-27 alone significantly increases the average weight of SMA mouse pups (+16%) relative to the control treatment with mismatch ASO. In combination with ASO 10-27, BIO101 induces a synergistic effect on the weight of SMA mouse pups with a significant increase of +30.9% at P10 (p<0.05), i.e. almost 2 times higher than that observed with ASO 10-27 alone.

This increase in body weight of mouse pups treated with ASO 10-27+BIO101 relative to that of mouse pups treated with ASO 10-27 alone continues until their death, with in particular a significant increase of 33% at P15 (p<0.05), 49.4% at P20 (p<0.05), 57.6% at P25 (p<0.01).

b. Analysis of the Functional Effects of the ASO 10-27+BIO101 Combined Treatment

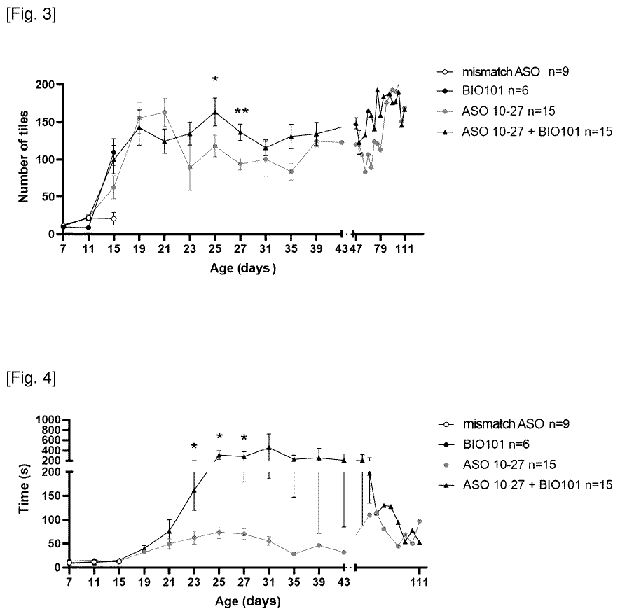

We performed phenotypic analyses of severe SMA type 2 mice treated with BIO101 alone or a mismatch ASO or ASO 10-27 alone, or with an ASO 10-27+BIO101 combined treatment from PO. We carried out a longitudinal monitoring of the motor abilities of mice. We, on the one hand, evaluated the movement capacities by the open-field test (FIG. 3), as well as the muscular fatigability by grip-test (FIG. 4), as previously described (Biondi et al., 2008; Branchu et al., 2013; Chali et al., 2016).

The device used for open-field testing consists of a plastic box measuring 28×28×5 cm with a field grid divided into 16 tiles measuring 7 cm×7 cm. Mice were tested individually and the evaluation device was washed after each session. Each mouse initially placed in the centre of the field has been able to move freely for 5 minutes with tail pinch stimulation every 15 seconds. The behavioural measurements were recorded by the experimenter during these 5 minutes and the total number of crossed tiles was recorded.

The BIO101 treatment, whether administered alone or in combination with ASO 10-27, accelerates the acquisition of walking with a tendency to increase the movement of the mice at P15 (110±18 tiles or +58% with the BIO101 treatment alone, and 100±19 tiles i.e. +44% with the ASO 10-27+BIO101 treatment) relative to the mouse pups treated with ASO 10-27 alone (70±20 tiles).

From P23, the movement capacities of mouse pups treated with ASO 10-27+BIO101 tend to be increased relative to those of mouse pups treated with ASO 10-27 alone (+51.3%, p=ns), and are significantly increased at P25 (+38.4%, p<0.05), or even at P27 (+44.9%, p<0.01) (FIG. 3).

In order to assess the muscle fatigability, the grip strength of the mice's forelimbs was tested. The mice are suspended by the front legs thereof from a thin metal rod which is horizontally suspended in the air. The time spent hanging on is recorded. Each mouse was subjected to five successive attempts with a one-minute rest period between two tests. Only the best test is kept for the evaluation of the muscle functions.

At P7 the resistance to fatigue of the mouse pups treated with ASO 10-27 alone was not improved relative to that of the mouse pups treated with the mismatch ASO. The fatigue resistance of mice treated with BIO101 alone was itself improved by 19.3%. At P15, the fatigue resistance of the mice treated with ASO 10-27 alone and with BIO 101 alone were both higher (+7.7%) than that of the mouse pups treated with mismatch ASO. Surprisingly and advantageously, a synergy operates between BIO101 and ASO 10-27 during the combined treatment such that the fatigue resistance of the treated SMA animals increases by 26.3% at P7 and by 21.2% at P15.

From P19, the fatigue resistance of mouse pups treated with ASO 10-27+BIO101 tends to increase relative to that of mice treated with ASO 10-27 alone. This difference becomes evident from P21 (+54% in the group treated with the ASO 10-27+BIO101 combination relative to ASO 10-27 alone, p=ns), then at P23 (+159.2%, p<0.05), at P25 (+337.7%, p<0.05) or even at P27 (+296.7%, p=0.07). These significant differences are maintained until P35. From P39, these differences are less marked but the group of mice treated with the ASO 10-27+BIO101 combination nevertheless maintains a resistance to fatigue which is greater than that observed in the group treated with ASO 10-27 alone (FIG. 4).

### 4. Conclusion

These results demonstrate the interest in using the BIO101 treatment, in combination with an active ingredient aimed at restoring the expression of the SMN protein, in particular thanks to a therapeutic approach using ASOs having the effect of restoring the expression of the SMN protein.

Indeed, this combined therapy shows the significant beneficial effects in a mouse model of SMA, in particular in terms of the weight of the animals, and even more significantly, in terms of the physical performance of these animals, whether movement capabilities or resistance to fatigue of the animal.

The combination of ASO 10-27+BIO101 treatment, beyond improving the performance obtained by the treatment of an ASO 10-27 as monotherapy, undoubtedly has a synergistic effect.

More generally, it should be noted that the implementations and embodiments of the invention which are considered above have been described by way of non-limiting examples and that other variants are therefore possible.

In particular, the invention has been described by mainly considering ASO 10-27 of sequence SEQ ID NO: 23. However, nothing excludes, in other types of embodiment, from considering other active ingredients having the ability to increase the production of SMN protein such as other ASOs having the ability to increase the production of SMN protein. Such ASOs are for example the ASOs of sequence selected from: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 25, SEQ ID NO: 26, SEQ ID NO: 27, SEQ ID NO: 28, SEQ ID NO: 29.

These sequences are given in particular in table 1 below:

TABLE 1

| | | Residues |
|---|---|---|
| | | targeted |
| | | by ASO in |
| | | the intron 7 |
| Sequence name | ASO sequence | of SMN2 |
| SEQ ID NO: 1 | TCACTTTCATAATGCTGG | 10 to 27 |
| SEQ ID NO: 2 | ACTTTCATAATGCTGGCA | 8 to 25 |
| SEQ ID NO: 3 | CACTTTCATAATGCTGGC | 9 to 26 |
| SEQ ID NO: 4 | TTCACTTTCATAATGCTG | 11 to 28 |
| SEQ ID NO: 5 | AGTAAGATTCACTTT | 21 to 35 |
| SEQ ID NO: 6 | GATTCACTTTCATAA | 16 to 30 |
| SEQ ID NO: 7 | ATTCACTTTCATAAT | 15 to 29 |
| SEQ ID NO: 8 | TTCACTTTCATAATG | 14 to 28 |
| SEQ ID NO: 9 | TCACTTTCATAATGC | 13 to 27 |
| SEQ ID NO: 10 | CACTTTCATAATGCT | 12 to 26 |
| SEQ ID NO: 11 | ACTTTCATAATGCTG | 11 to 25 |
| SEQ ID NO: 12 | CTTTCATAATGCTGG | 10 to 24 |
| SEQ ID NO: 13 | TTTCATAATGCTGGC | 9 to 23 |
| SEQ ID NO: 14 | TTCATAATGCTGGCA | 8 to 22 |
| SEQ ID NO: 15 | TCATAATGCTGGCAG | 7 to 21 |
| SEQ ID NO: 16 | CATAATGCTGGCAGA | 6 to 20 |
| SEQ ID NO: 17 | TGCTGGCAGACTTAC | 1 to 15 |
| SEQ ID NO: 18 | ATTCACTTTCAT | 18 to 29 |
| SEQ ID NO: 19 | TTCACTTTCATA | 17 to 28 |
| SEQ ID NO: 20 | TCACTTTCATAA | 16 to 27 |
| SEQ ID NO: 21 | CACTTTCATAAT | 15 to 26 |
| SEQ ID NO: 22 | ACTTTCATAATG | 14 to 25 |
| SEQ ID NO: 25 | CTTTCATAATGC | 13 to 24 |
| SEQ ID NO: 26 | TTTCATAATGCT | 12 to 23 |
| SEQ ID NO: 27 | TTCATAATGCTG | 11 to 22 |
| SEQ ID NO: 28 | TCATAATGCTGG | 10 to 21 |
| SEQ ID NO: 29 | ATTCACTTTCATAATGCTGG | 10 to 29 |

1. At least one phytoecdysone and/or at least one semi-synthetic derivative of 20-hydroxyecdysone, and at least one active ingredient having the ability to increase the production of functional SMN protein, for use in the treatment of spinal muscular atrophy in mammals.

2. At least one phytoecdysone and/or at least one semi-synthetic derivative of 20-hydroxyecdysone, and at least one active ingredient having the ability to increase the production of functional SMN protein for the use thereof according to claim 1, for use in the treatment of a motor neuron disease responsible for spinal muscular atrophy.

3. At least one phytoecdysone and/or at least one semi-synthetic derivative of 20-hydroxyecdysone, and at least one active ingredient having the ability to increase the production of functional SMN protein for the use thereof according to claim 2, wherein the motor neuron disease is an alteration in the function of the motor neurons or the degeneration thereof.

4. At least one phytoecdysone and/or at least one semi-synthetic derivative of 20-hydroxyecdysone, and at least one active ingredient having the ability to increase the production of functional SMN protein for the use thereof according to any one of claims 1 to 3, wherein the active ingredient has the ability to increase the production of functional SMN protein by gene therapy or by therapy of the gene.

5. At least one phytoecdysone and/or at least one semi-synthetic derivative of 20-hydroxyecdysone, and at least one active ingredient having the ability to increase the production of functional SMN protein for the use thereof according to any one of claims 1 to 3, wherein the active ingredient has the ability to increase the production of functional SMN protein by providing the SMN1 gene or by acting on the maturation of the SMN2 gene.

6. At least one phytoecdysone and/or at least one semi-synthetic derivative of 20-hydroxyecdysone, and at least one active ingredient having the ability to increase the production of functional SMN protein for the use thereof according to any one of claims 1 to 3, wherein the active ingredient has the ability to increase the production of functional SMN protein is an antisense oligonucleotide.

7. At least one phytoecdysone and/or at least one semi-synthetic derivative of 20-hydroxyecdysone, and at least one active ingredient having the ability to increase the production of functional SMN protein for the use thereof according to claim 6, wherein the antisense oligonucleotide includes 10 to 30 nucleotides.

8. At least one phytoecdysone and/or at least one semi-synthetic derivative of 20-hydroxyecdysone, and at least one active ingredient having the ability to increase the production of functional SMN protein for the use thereof according to any one of claims 6 to 7, wherein the antisense oligonucleotide is complementary to at least 90%, preferably at least 95%, most preferably at least 98%, preferably 100%, of the sequence of the nucleic acid encoding the pre-mRNA of the human SMN2 gene.

9. At least one phytoecdysone and/or at least one semi-synthetic derivative of 20-hydroxyecdysone, and at least one active ingredient having the ability to increase the production of functional SMN protein for the use thereof according to any one of claims 6 to 8, wherein the antisense oligonucleotide is complementary to at least 90%, preferably at least 95%, most preferably 98%, preferably 100%, of a sequence belonging to intron 6, intron 7 or exon 7 of the nucleic acid encoding the pre-mRNA of the human SMN2 gene.

10. At least one phytoecdysone and/or at least one semi-synthetic derivative of 20-hydroxyecdysone, and at least one active ingredient having the ability to increase the production of functional SMN protein for the use thereof according to any one of claims 6 to 9, wherein the antisense oligonucleotide is a sequence which is identical or similar to at least 50% with the sequence SEQ ID NO: 1, preferably identical or similar to at least 60%, preferably to at least 70%, more preferably to at least 80%, most preferably to at least 90% and preferably identical or similar to 100%.

11. At least one phytoecdysone and/or at least one semi-synthetic derivative of 20-hydroxyecdysone, and at least one active ingredient having the ability to increase the production of functional SMN protein for the use thereof according to any one of claims 6 to 9, wherein the ASO has a sequence selected from the sequences SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 25, SEQ ID NO: 26, SEQ ID NO: 27, SEQ ID NO: 28, SEQ ID NO: 29.

12. At least one phytoecdysone and/or at least one semi-synthetic derivative of 20-hydroxyecdysone, and at least one active ingredient having the ability to increase the production of functional SMN protein for the use thereof according to any one of claims 6 to 11, wherein the ASO includes at least one modification selected from:
a modification at the phosphate group such as a phosphorothioate, or a methylphosphanate, or a phosphoroamidate,
a chemical modification in the 2′ position of the ribose, such as a 2′O-methyl (2′OMe) or a 2′O-methoxyethyl (2′MOE) or a 2′ Fluoro (2′ F),
at least one modification of nucleobases such as the methylation of 5′ methylcytosine, 5-methyluridine/ribothymidine, or “G-clamp” type pyrimidine,
at least one substantial change in the sugar structure, leading to a variety of molecules, such as morpholinos or the peptide nucleic acids or oligonucleotides of constrained type.

13. At least one phytoecdysone and/or at least one semi-synthetic derivative of 20-hydroxyecdysone, and at least one active ingredient having the ability to increase the production of functional SMN protein for the use thereof according to any one of claims 6 to 9, wherein the antisense oligonucleotide is a sequence which is identical or similar to at least 50% with the sequence SEQ ID NO: 23, preferably identical or similar to at least 60%, preferably at least 70%, more preferably at least 80%, most preferably at least 90% and preference identical or similar to 100%.

14. At least one phytoecdysone and/or at least one semi-synthetic derivative of 20-hydroxyecdysone, and at least one active ingredient having the ability to increase the production of functional SMN protein for the use thereof according to any one of claims 1 to 13, wherein said at least one phytoecdysone is 20-hydroxyecdysone.

15. At least one phytoecdysone and/or at least one semi-synthetic derivative of 20-hydroxyecdysone, and at least one active ingredient having the ability to increase the production of functional SMN protein for the use thereof according to claim 14, wherein 20-hydroxyecdysone is in the form of a plant extract or a plant portion, said extract including at least 95%, and preferably at least 97%, of 20-hydroxyecdysone.

16. At least one phytoecdysone and/or at least one semi-synthetic derivative of 20-hydroxyecdysone, and at least one active ingredient having the ability to increase the production of functional SMN protein for the use thereof according to any one of claims 1 to 15, wherein said at least one semi-synthetic derivative of 20-hydroxyecdysone is selected from:
a compound of general formula (I): 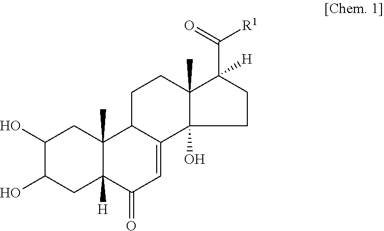
wherein:
R¹ is selected from: a (C₁-C₆) W(C₁-C₆) group; a (C₁-C₆) W(C₁-C₆) W(C₁-C₆) group; a (C₁-C₆) W(C₁-C₆) CO₂ (C₁-C₆) group; a (C₁-C₆) A group, A representing a heterocycle optionally substituted by a group of the OH, OMe, (C₁-C₆), N(C₁-C₆), CO₂ (C₁-C₆) type; a CH₂Br group;
W being a heteroatom selected from N, O and S, preferably O and even more preferably S°; and,
a compound having formula (II):

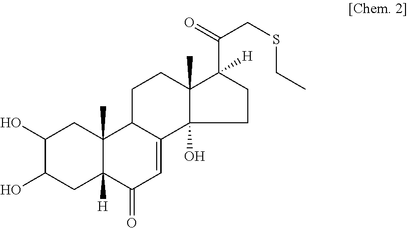 [SMILES: CCSCC(=O)[C@H]1CC[C@@]2(O)C3=CC(=O)[C@@H]4CC(O)C(O)C[C@]4(C)C3CC[C@]12C]

17. At least one phytoecdysone and/or at least one semi-synthetic derivative of 20-hydroxyecdysone, and at least one active ingredient having the ability to increase the production of functional SMN protein for the use thereof according to claim 16, wherein in general formula (I):
R¹ is selected from: a (C₁-C₆) W(C₁-C₆) group; a (C₁-C₆) W(C₁-C₆) W(C₁-C₆) group; a (C₁-C₆) W(C₁-C₆) CO₂ (C₁-C₆) group; a (C₁-C₆) A group, A representing a heterocycle optionally substituted by a group of the OH, OMe, (C₁-C₆), N(C₁-C₆), CO₂ (C₁-C₆) type;
W being a heteroatom selected from N, O and S, preferably O and more preferably S.

18. At least one phytoecdysone and/or at least one semi-synthetic derivative of 20-hydroxyecdysone, and at least one active ingredient having the ability to increase the production of functional SMN protein for the use thereof according to any one of claims 16 to 17, wherein at least one compound of general formula (I) is selected from:
no. 1: (2S,3R,5R,10R,13R,14S, 17S)-2,3,14-trihydroxy-10,13-dimethyl-17-(2-morpholinoacetyl)-2,3,4,5,9,11,12,15,16,17-decahydro-1H-cyclopenta[a]phenanthren-6-one,
no. 2: (2S,3R,5R,10R,13R,14S,17S)-2,3,14-trihydroxy-17-[2-(3-hydroxypyrrolidin-1-yl) acetyl]-10,13-dimethyl-2,3,4,5,9,11,12,15,16,17-decahydro-1H-cyclopenta[a]phenanthren-6-one;
no. 3: (2S,3R,5R,10R,13R,14S,17S)-2,3,14-trihydroxy-17-[2-(4-hydroxy-1-piperidyl) acetyl]-10,13-dimethyl-2,3,4,5,9,11,12,15,16,17-decahydro-1H-cyclopenta[a]phenanthren-6-one;
no. 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
no. 5: (2S,3R,5R,10R,13R,14S, 17S)-17-[2-(3-dimethylaminopropyl (methyl)amino) acetyl]-2,3,14-trihydroxy-10,13-dimethyl-2,3,4,5,9,11,12,15,16,17-decahydro-1H-cyclopenta[a]phenanthren-6-one;
no. 6:2-[2-oxo-2-[(2S,3R,5R,10R, 13R,14S,17S)-2,3,14-trihydroxy-10,13-dimethyl-6-oxo-2,3,4,5,9,11,12,15,16,17-décahydro-1H-cyclopenta[a]phenanthren-17-yl]ethyl]ethylsulfanylacetate;
no. 7: (2S,3R,5R,10R,13R,14S, 17S)-17-(2-ethylsulfanylacetyl)-2,3,14-trihydroxy-10,13-dimethyl-2,3,4,5,9,11,12,15,16,17-decahydro-1H-cyclopenta[a]phenanthren-6-one;
no. 8: (2S,3R,5R,10R,13R,14S,17S)-2,3,14-trihydroxy-17-[2-(2-hydroxyethyl sulfanyl) acetyl]-10,13-dimethyl-2,3,4,5,9,11,12,15,16,17-decahydro-1H cyclopenta[a]phenanthren-6-one.

19. Composition comprising:
at least one phytoecdysone and/or at least one semi-synthetic derivative of 20-hydroxyecdysone,
at least one active ingredient having the ability to increase the production of functional SMN protein,
for the use thereof in the treatment of spinal muscular atrophy in mammals.


SEQUENCE LISTING (29 sequences)

SEQ ID NO: 1 - DNA, 18 bp, synthetic construct
  source 1..18: mol_type = other DNA
tcactttcataatgctgg

SEQ ID NO: 2 - DNA, 18 bp, synthetic construct
  source 1..18: mol_type = other DNA
actttcataatgctggca

SEQ ID NO: 3 - DNA, 18 bp, synthetic construct
  source 1..18: mol_type = other DNA
cactttcataatgctggc

SEQ ID NO: 4 - DNA, 18 bp, synthetic construct
  source 1..18: mol_type = other DNA
ttcactttcataatgctg

SEQ ID NO: 5 - DNA, 15 bp, synthetic construct
  source 1..15: mol_type = other DNA
agtaagattcacttt

SEQ ID NO: 6 - DNA, 15 bp, synthetic construct
  source 1..15: mol_type = other DNA
gattcactttcataa

SEQ ID NO: 7 - DNA, 15 bp, synthetic construct
  source 1..15: mol_type = other DNA
attcactttcataat

SEQ ID NO: 8 - DNA, 15 bp, synthetic construct
  source 1..15: mol_type = other DNA
ttcactttcataatg

SEQ ID NO: 9 - DNA, 15 bp, synthetic construct
  source 1..15: mol_type = other DNA
tcactttcataatgc

SEQ ID NO: 10 - DNA, 15 bp, synthetic construct
  source 1..15: mol_type = other DNA
cactttcataatgct

SEQ ID NO: 11 - DNA, 15 bp, synthetic construct
  source 1..15: mol_type = other DNA
actttcataatgctg

SEQ ID NO: 12 - DNA, 15 bp, synthetic construct
  source 1..15: mol_type = other DNA
ctttcataatgctgg

SEQ ID NO: 13 - DNA, 15 bp, synthetic construct
  source 1..15: mol_type = other DNA
tttcataatgctggc

SEQ ID NO: 14 - DNA, 15 bp, synthetic construct
  source 1..15: mol_type = other DNA
ttcataatgctggca

SEQ ID NO: 15 - DNA, 15 bp, synthetic construct
  source 1..15: mol_type = other DNA
tcataatgctggcag

SEQ ID NO: 16 - DNA, 15 bp, synthetic construct
  source 1..15: mol_type = other DNA
cataatgctggcaga

SEQ ID NO: 17 - DNA, 15 bp, synthetic construct
  source 1..15: mol_type = other DNA
tgctggcagacttac

SEQ ID NO: 18 - DNA, 12 bp, synthetic construct
  source 1..12: mol_type = other DNA
attcactttcat

SEQ ID NO: 19 - DNA, 12 bp, synthetic construct
  source 1..12: mol_type = other DNA
ttcactttcata

SEQ ID NO: 20 - DNA, 12 bp, synthetic construct
  source 1..12: mol_type = other DNA
tcactttcataa

SEQ ID NO: 21 - DNA, 12 bp, synthetic construct
  source 1..12: mol_type = other DNA
cactttcataat

SEQ ID NO: 22 - DNA, 12 bp, synthetic construct
  source 1..12: mol_type = other DNA
actttcataatg

SEQ ID NO: 23 - DNA, 18 bp, synthetic construct
  modified_base 2: mod_base = m5c
  modified_base 4: mod_base = m5c
  modified_base 8: mod_base = m5c
  modified_base 15: mod_base = m5c
  source 1..18: mol_type = other DNA
  modified_base 1..18: mod_base = OTHER; note = Phosphorothioate backbone obtained by replacing the phosphodiester (PO) by a phosphorothioate group (PS)
  modified_base 1..18: mod_base = OTHER; note = 2'-O-methoxyethyl nucleosides
tcactttcataatgctgg

SEQ ID NO: 24 - DNA, 18 bp, synthetic construct
  source 1..18: mol_type = other DNA
ttagtttaatcacgctcg

SEQ ID NO: 25 - DNA, 12 bp, synthetic construct
  source 1..12: mol_type = other DNA
ctttcataatgc

SEQ ID NO: 26 - DNA, 12 bp, synthetic construct
  source 1..12: mol_type = other DNA
tttcataatgct

SEQ ID NO: 27 - DNA, 12 bp, synthetic construct
  source 1..12: mol_type = other DNA
ttcataatgctg

SEQ ID NO: 28 - DNA, 12 bp, synthetic construct
  source 1..12: mol_type = other DNA
tcataatgctgg

SEQ ID NO: 29 - DNA, 20 bp, synthetic construct
  source 1..20: mol_type = other DNA
attcactttcataatgctgg